(19) United States
(12) Patent Application Publication
(10) Pub. No.: US 2022/0339307 A1
(43) Pub. Date: Oct. 27, 2022

(54) PLANAR LIGHTING STERILIZATION APPARATUS

(71) Applicants: Chang Sin LEE, Busan (KR); CONNETIVITY & INTELLIGENCE SYSTEM CO., LTD., Busan (KR)

(72) Inventors: Chang Sin LEE, Busan (KR); Chol Han KIM, Yangsan-si, Gyeongsangnam-do (KR); Byung Tae KIM, Changwon-si, Gyeongsangnam-do (KR); Ji Won KIM, Busan (KR); Boum KIM, Busan (KR)

(21) Appl. No.: 17/641,375

(22) PCT Filed: May 20, 2021
(86) PCT No.: PCT/KR2021/006266

§ 371 Date: Mar. 8, 2022

(30) Foreign Application Priority Data
Jul. 22, 2020 (KR) 10-2020-0090974

(51) Int. Cl. A61L 2/10; A61L 2/24

(52) U.S. Cl. CPC A61L 2/10; A61L 2/24; A61L 2202/11; A61L 2202/14

(57) ABSTRACT

Disclosed herein is a sterilizer with planar lighting. The sterilizer with planar lighting can perform sterilization in a safety-secured state to maintain a residential/office space in a sanitary state by automatically turning on a sterilization lamp only when no object such as a person or a companion animal is present in an illumination target region. It is also possible to perform power supply control and operation control according to various environmental conditions and situations by connecting a planar luminaire and a sterilization lamp to an SMPS through a switching operation unit and allowing the switching operation unit to selectively perform simultaneous power supply to the planar luminaire and the sterilization lamp, individual power supply thereto, connection with a central control server, individual connection with a luminaire switch/sterilization lamp switch, etc.

### TECHNICAL FIELD

The present invention relates to a sterilizer with planar lighting, and more particularly, to a sterilizer with planar lighting, which is configured to combine a sterilization lamp with a planar luminaire provided in a residential or office space and to detect an object such as a person or a companion animal, thereby sterilizing the residential or office space in a time period during which no object is recognized.

### BACKGROUND ART

In recent years, rapid development of vehicles has led to a rapid increase in foreign travel culture as well as active exchange between countries due to freedom of movement, which is a major cause of the spread of infectious diseases. In other words, there are frequent outbreaks of global pandemics, such as MERS, H1N1 flu, SARS, and COVID-19, in which infectious viruses in a specific region contaminate other regions.

With increasing global awareness of these global pandemics, a new service that has made virus sterilization a daily routine is required. In particular, there is a need for a service that prevents the spread of viruses by periodically sterilizing a building where people come and go frequently, an entrance to a residential area, or a space for placement of daily items such as a shoe rack or a wardrobe.

Ultraviolet (UV) light is typically utilized for sterilizing daily items. Especially, UV-C with a wavelength of 100 to 280 nm has excellent sterilizing power capable of sterilizing 99.99% of viruses even in a short time. However, UV sterilization may be harmful to the human body, such as causing skin damage or aging.

Moreover, no sterilizer in a modular form has been developed yet. In order to conventionally install a sterilizer in a residential or office space, the sterilizer may have to be connected to a power source by means of separate wiring. The sterilizer may have to be fixed with nails, adhesives, or the like.

### DISCLOSURE

### Technical Problem

Therefore, the present invention has been made in view of the above problems, and it is an object of the present invention to provide a new type of sterilizer with planar lighting, which is configured to organically combine a sterilization lamp emitting ultraviolet light with a planar luminaire commonly placed to illuminate a residential or office space.

It is another object of the present invention to provide a new type of sterilizer with planar lighting, which is capable of performing sterilization in a safety-secured state to maintain a residential/office space in a sanitary state by automatically turning on a sterilization lamp only when no object such as a person or a companion animal is present in an illumination target region.

It is a further object of the present invention to provide a new type of sterilizer with planar lighting, which is capable of performing power supply control and operation control according to various environmental conditions and situations by connecting a planar luminaire and a sterilization lamp to an SMPS through a switching operation unit and allowing the switching operation unit to selectively perform simultaneous power supply to the planar luminaire and the sterilization lamp, individual power supply thereto, connection with a central control server, individual connection with a luminaire switch/sterilization lamp switch, etc.

### Technical Solution

In accordance with the present invention, the above and other objects can be accomplished by the provision of a sterilizer with planar lighting, which includes a planar luminaire (100) configured to irradiate an illumination target region (2) with light by exposing a light projection surface (110) having a set area, a sterilization lamp (200) configured to perform sterilization capability by irradiating the illumination target region (2) with ultraviolet light, a power supply unit (300) configured to provide electric power to operate the planar luminaire (100) the sterilization lamp (200), a PIR sensor unit (400) configured to perform real-time infrared detection of the illumination target region (2) and to generate object recognition information indicating that a person or an animal is present in the illumination target region (2), an illumination-sterilization control unit (500) connected to the planar luminaire (100), the sterilization lamp (200), and the PIR sensor unit (400), and configured to control operation of the sterilization lamp (200) by an automatic sterilization process when the object recognition information is not input from the PIR sensor unit (400) and to deactivate the sterilization lamp (200) by a sterilization stop process when the object recognition information is input from the PIR sensor unit (400), and a sterilization lamp-sensor assembly (1200). The sterilization lamp-sensor assembly (1200) includes a lower housing (1220) fixedly inserted into a mounting space for fitting (1110) of a frame (1100) formed integrally with the planar luminaire (100) around the planar luminaire (100), the mounting space (1110) being recessed to a set depth, the frame (1100) having two parallel sides, an upper housing (1210) detachably coupled to the lower housing (1220) and having an open surface (1211) and a sensor through-hole (1212) formed on an upper surface thereof exposed to the outside, an LED-sensor substrate (1250) fixed inside the lower housing (1220), in which case a plurality of sterilization LEDs (210) are arranged beneath the open surface (1211) of the upper housing (1210) and the PIR sensor unit (400) is disposed beneath the sensor through-hole (1212), and a quartz glass plate (1260) disposed in communication with the open surface (1211) of the upper housing (1210) to transmit ultraviolet light from the sterilization LEDs (210), the sterilization lamp-sensor assembly (1200) being fitted in a detachable assembly structure into the mounting space (1110). The PIR sensor unit (400) consists of a PIR sensor unit (400), which is installed on one side of the frame and includes a first sensor (410a) having a structure in which a pair of elongated straight pyroelectric sensor elements (411) are spaced parallel to each other and connected in series, and a PIR sensor unit (400) which is installed on the other side of the frame and includes a second sensor (410b) having a pair of pyroelectric sensor elements (411) formed parallel to each other in a direction perpendicular to the installation direction of the pyroelectric sensor elements (411) of the first sensor (410a).

The illumination-sterilization control unit (500) may include a mode selection management module (510) configured to select one from a group consisting of a daily resident mode, a non-resident mode, an office operation mode, and an office non-operation mode by an administrator, a daily resident mode sterilization management module (520) configured to turn on the sterilization lamp (200) after a set delay time from a start time at which the object recognition information is not input in a time period during which the object recognition information is not input in the daily resident mode, and to turn off the sterilization lamp (200) after a set sterilization duration time has elapsed, the daily resident mode sterilization management module (520) being configured to immediately turn off the sterilization lamp (200) when the object recognition information is input while the sterilization lamp (200) is in operation, a non-resident mode sterilization management module (530) configured to turn on/off the sterilization lamp (200) at a set sterilization cycle in a time period in which the object recognition information is not input in the non-resident mode, the non-resident mode sterilization management module (530) being configured to immediately turn off the sterilization lamp (200) when the object recognition information is input while the sterilization lamp (200) is in operation, the sterilization cycle being set by the administrator, an office operation mode sterilization management module (540) configured to allow the sterilization lamp (200) to remain off in the office operation mode, and an office non-operation mode sterilization management module (550) configured to turn on/off the sterilization lamp (200) at a set sterilization cycle in a time period in which the object recognition information is not input in the office non-operation mode, the office non-operation mode sterilization management module (550) being configured to immediately turn off the sterilization lamp (200) when the object recognition information is input while the sterilization lamp (200) is in operation, the sterilization cycle being set by the administrator.

The sterilizer with planar lighting may include an SMPS (600) connected to the power supply unit (300), a switching operation unit (700) connected to the SMPS (600), and configured to implement a plurality of switching operation modes for applying electric power to the planar luminaire (100) and the sterilization lamp (200), and a power control unit (800) configured to control implementation of the switching operation modes of the switching operation unit (700). The switching operation unit (700) may include a simultaneous connection switching module (710) configured to induce anti-virus planar lighting by connecting the planar luminaire (100) and the sterilization lamp (200) to the SMPS (600) at the same time, and an individual connection switching module (720) configured to connect each of the planar luminaire (100) and the sterilization lamp (200) to the SMPS (600) individually, to induce implementation of planar lighting capability or sterilization capability by user's arbitrary selection.

The sterilizer with planar lighting may further include a rechargeable battery (1000) connected to the power supply unit (300) for charging and connected to the sterilization lamp (200) for supply of electric power thereto, and a power control unit (800) connected to the power supply unit (300) and the rechargeable battery (1000) to control power supply, the power control unit (800) being configured, when the planar luminaire (100) is turned on, to induce charging of the rechargeable battery (1000) while cutting off supply of electric power to the sterilization lamp (200) from the rechargeable battery (1000) and the power supply unit (300), the power control unit (800) being configured, when the planar luminaire (100) is turned off, to apply electric power to the sterilization lamp (200) from one of the rechargeable battery (1000) and the power supply unit (300). The sterilizer with planar lighting may allow the sterilization lamp (200) to be turned on after the planar luminaire (100) is turned off, regardless of operation of a luminaire switch (3) and a sterilization lamp switch (4).

### Advantageous Effects

According to a sterilizer with planar lighting of the present invention, it is possible to perform sterilization in a safety-secured state to maintain a residential/office space in a sanitary state by automatically turning on a sterilization lamp only when no object such as a person or a companion animal is present in an illumination target region. According to the sterilizer with planar lighting of the present invention, it is possible to perform power supply control and operation control according to various environmental conditions and situations by connecting a planar luminaire and a sterilization lamp to an SMPS through a switching operation unit and allowing the switching operation unit to selectively perform simultaneous power supply to the planar luminaire and the sterilization lamp, individual power supply thereto, connection with a central control server, individual connection with a luminaire switch/sterilization lamp switch, etc.

### DESCRIPTION OF DRAWINGS

FIG. 1 is a block diagram illustrating a sterilizer with planar lighting according to an embodiment of the present invention.

FIG. 2 is a view illustrating main components of the sterilizer with planar lighting according to the embodiment of the present invention.

FIGS. 3 to 6 are views illustrating a structure of a sterilization lamp-sensor assembly applied to the sterilizer with planar lighting according to the embodiment of the present invention.

FIGS. 7 to 10 are diagrams illustrating power supply control and power supply operation control of the sterilizer with planar lighting according to the embodiment of the present invention.

FIGS. 11 to 14 are views illustrating a detailed configuration of a PIR sensor unit according to the embodiment of the present invention.

FIG. 15 is a block diagram illustrating an illumination-sterilization control unit according to the embodiment of the present invention.

### BEST MODE

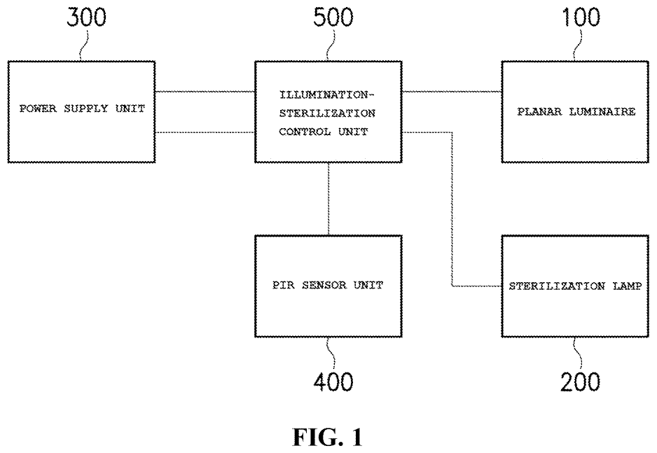

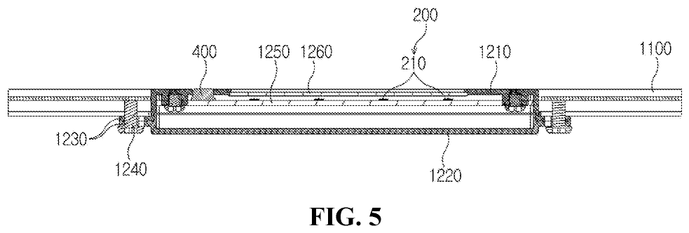

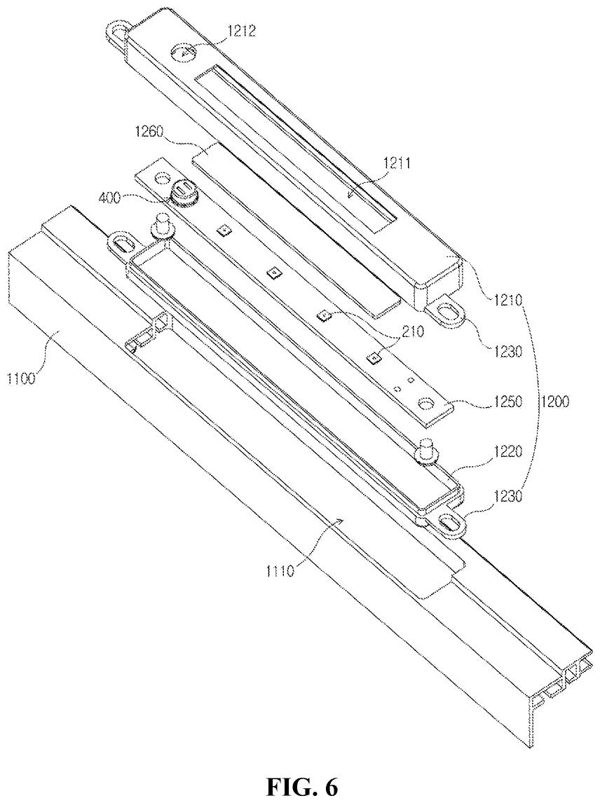

Hereinafter, an exemplary embodiment of the present invention will be described in detail with reference to the accompanying drawings (FIGS. 1 to 15). Meanwhile, in the drawings and specification, configurations and operations that can be easily understood by those skilled in the art will be briefly illustrated or omitted. In particular, in the drawings and specification, illustrations and descriptions of technical configurations and operations directly unrelated to the technical features of the present invention are omitted, and only the technical configurations related to the present invention will be briefly illustrated or described.

The sterilizer with planar lighting, which is designated by reference numeral 1, according to the embodiment of the present invention includes a planar luminaire 100, a sterilization lamp 200, a power supply unit 300, a PIR sensor unit 400, and an illumination-sterilization control unit 500, as illustrated in FIG. 1.

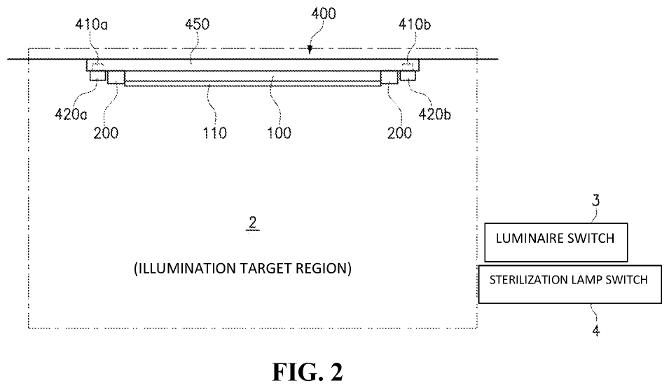

As illustrated in FIG. 2, the planar luminaire 100 irradiates an illumination target region 2 with light by exposing a light projection surface 110 having a set area, and may use various lighting fixtures including LEDs and fluorescent lamps. The sterilization lamp 200 performs sterilization capability by irradiating the illumination target region 2 with ultraviolet light. Especially, the sterilization lamp 200 according to the embodiment of the present invention emits UV-C for an improvement in sterilization capability.

The planar luminaire 100 and the sterilization lamp 200 may be fixedly installed on a wall surface (ceiling surface, etc.) of a residential or office space, or may be fixedly installed on a separate post. In addition, the planar luminaire 100 and the sterilization lamp 200 may be combined and integrated with each other, or may be disposed separately from each other.

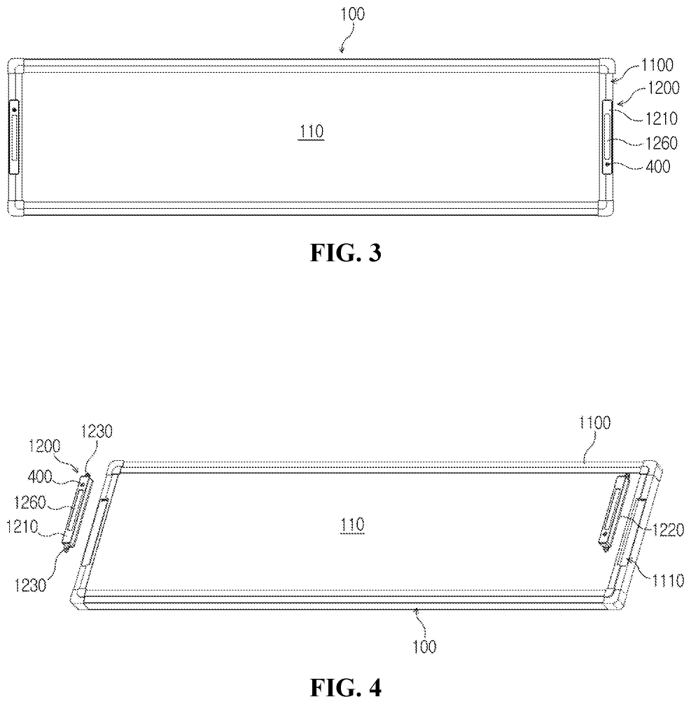

The sterilizer with planar lighting 1 according to the embodiment of the present invention may include a sterilization lamp-sensor assembly 1200 mounted in a detachable assembly structure on a frame 1100 which is formed integrally with the planar luminaire 100 around the planar luminaire 100, as illustrated in FIGS. 3 to 6.

The frame 1100 has a mounting space 1110 into which the sterilization lamp-sensor assembly 1200 is inserted and fitted to a set depth. The frame 1100 may be disposed on the entire circumference of the planar luminaire 100, or may be disposed on the left and right portions of the planar luminaire 100.

The sterilization lamp-sensor assembly 1200 may include a lower housing 1220, an upper housing 1210, a plurality of sterilization LEDs 210, a PIR sensor unit 400, an LED-sensor substrate 1250, and a quartz glass plate 1260.

The lower housing 1220 and the upper housing 1210 are coupled to each other to define an external appearance of the sterilization lamp-sensor assembly 1200. The lower housing 1220 is fixedly inserted into the mounting space 1110 of the frame 1100, and the upper housing 1210 is detachably coupled to the lower housing 1220 and has a portion exposed to the outside. The upper housing 1210 has an open surface 1211 and a sensor through-hole 1212, which are formed on the upper surface thereof exposed to the outside. The lower housing 1220 and the upper housing 1210 each have connection pieces 1230 formed at both ends thereof, and are fixed to the frame 1100 while being fastened to each other by fastening bolts 1240 passing through the connection pieces 1230.

As illustrated in FIG. 4, the sterilization lamp-sensor assembly 1200 is formed on each of two parallel sides of the frame 1100. In the following description, the two sides will be given as a left frame and a right frame.

The sterilization LEDs 210 constitute the sterilization lamp 200, and emit ultraviolet light.

The PIR sensor unit 400 includes one of a first sensor 410a and a second sensor 410b, each having a structure in which a pair of elongated straight pyroelectric sensor elements 411 are spaced parallel to each other and connected in series. The pyroelectric sensor elements 411 of the first sensor 410a disposed on the left frame of the planar luminaire 100 are placed in an elongated straight shape in a reference direction, and the pyroelectric sensor elements 411 of the second sensor 410b disposed on the right frame of the planar luminaire 100 is placed in an elongated straight shape in a direction perpendicular to the reference direction.

Since the pyroelectric sensor elements 411 each have a larger sensing response to the variation in the longitudinal direction thereof, an object moving at right angles to the direction of parallel installation of the pyroelectric sensor elements 411 has a maximum response characteristic.

Accordingly, for example, arranging the pyroelectric sensor elements 411 parallel to X and Y axes and at right angles to each other in an X-Y coordinate plane maximizes efficiency of sensing of movement in the X-Y coordinate plane.

The LED-sensor substrate 1250 is fixed to the lower housing 1220, and the sterilization LEDs 210 are fixedly arranged on the LED-sensor substrate 1250 while the PIR sensor unit 400 is fixedly disposed at a set position of the LED-sensor substrate 1250. The sterilization LEDs 210 are spaced in a line at set intervals on the LED-sensor substrate 1250 to induce uniform emission of ultraviolet light. The PIR sensor unit 400 fixed to the LED-sensor substrate 1250 is exposed to the outside through the sensor through-hole 1212 of the upper housing 1210.

The quartz glass plate 1260 is disposed in communication with the open surface 1211 of the upper housing 1210 to transmit ultraviolet light from the sterilization LEDs 210.

The power supply unit 300 provides electric power to operate the planar luminaire 100 and the sterilization lamp 200.

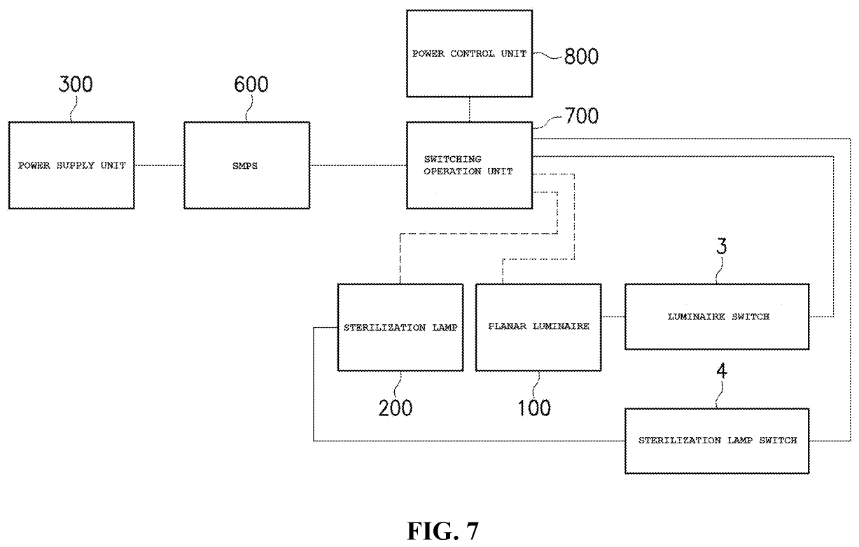

The sterilizer with planar lighting 1 according to the embodiment of the present invention includes a switched mode power supply (SMPS) 600, a switching operation unit 700, and a power control unit 800, and may operate in conjunction with a central control server 900, as illustrated in FIGS. 7 to 9.

The SMPS 600 is connected to the power supply unit 300, and is driven by a switching control method that converts AC power into DC power using a switching transistor or the like.

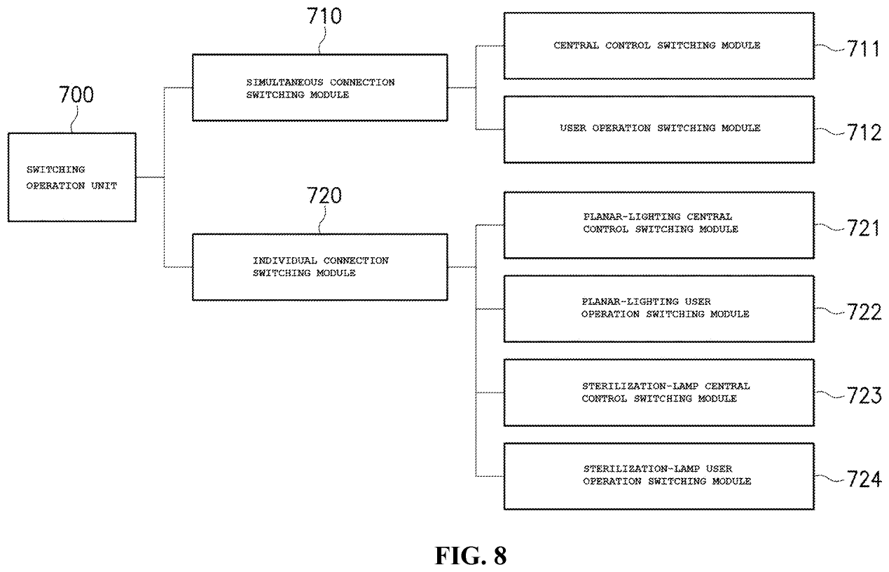

The switching operation unit 700 is connected to the SMPS 600, and implements a plurality of switching operation modes for applying electric power to the planar luminaire 100 and the sterilization lamp 200. The switching operation unit 700 according to the embodiment of the present invention includes a simultaneous connection switching module 710 and an individual connection switching module 720 as illustrated in FIG. 8.

The simultaneous connection switching module 710 serves to induce anti-virus planar lighting by connecting the planar luminaire 100 and the sterilization lamp 200 to the SMPS 600 at the same time. The simultaneous connection switching module 710 according to the embodiment of the present invention includes a central control switching module 711 and a user operation switching module 712.

The central control switching module 711 connects the planar luminaire 100 and the sterilization lamp 200 to the central control server 900 at the same time, to induce central operation control for the planar luminaire 100 and the sterilization lamp 200. The user operation switching module 712 connects the planar luminaire 100 and the sterilization lamp 200 to a luminaire switch 3 and a sterilization lamp switch 4 provided in the illumination target region 2 at the same time, to induce user operation control for the planar luminaire 100 and the sterilization lamp 200.

The individual connection switching module 720 connects each of the planar luminaire 100 and the sterilization lamp 200 to the SMPS 600 individually, to induce implementation of planar lighting capability or sterilization capability by user's arbitrary selection. The individual connection switching module 720 according to the embodiment of the present invention includes a planar-lighting central control switching module 721, a planar-lighting user operation switching module 722, a sterilization-lamp central control switching module 723, a sterilization-lamp user operation switching module 724.

The planar-lighting central control switching module 721 connects the planar luminaire 100 to the central control server 900 individually, to induce central operation control for the planar luminaire 100. The planar-lighting user operation switching module 722 connects the planar luminaire 100 to the luminaire switch 3 provided in the illumination target region 2 individually, to induce user operation control for the planar luminaire 100. The sterilization-lamp central control switching module 723 connects the sterilization lamp 200 to the central control server 900 individually, to induce central operation control for the sterilization lamp 200. The sterilization-lamp user operation switching module 724 connects the sterilization lamp 200 to the sterilization lamp switch 4 provided in the illumination target region 2 individually, to induce user operation control for the sterilization lamp 200.

The power control unit 800 controls implementation of switching operation modes of the switching operation unit 700. The power control unit 800 according to the embodiment of the present invention controls implementation of a simultaneous connection switching operation mode (a central control switching operation mode and a user operation switching operation mode) and an individual connection switching operation mode (a planar-lighting central control switching operation mode, a planar-lighting user operation switching operation mode, a sterilization-lamp central control switching operation mode, and a sterilization-lamp user operation switching operation mode).

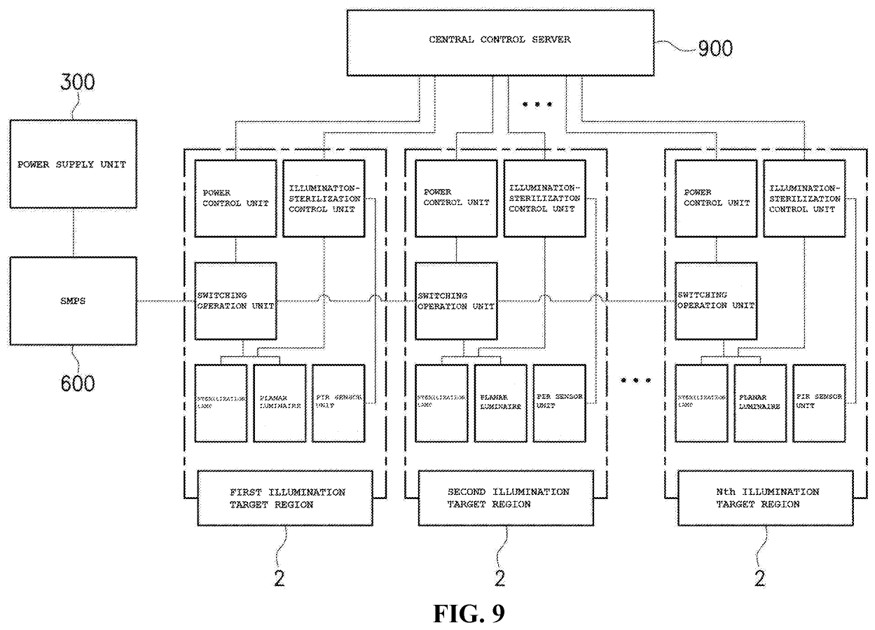

The central control server 900 is a server that performs central control of a plurality of planar luminaires 100 and sterilization lamps 200 installed in a plurality of illumination target regions 2 located in the residential and office spaces as illustrated in FIG. 9 when the sterilizer with planar lighting 1 according to the embodiment of the present invention is operated in the central control switching operation mode of the simultaneous connection switching operation mode.

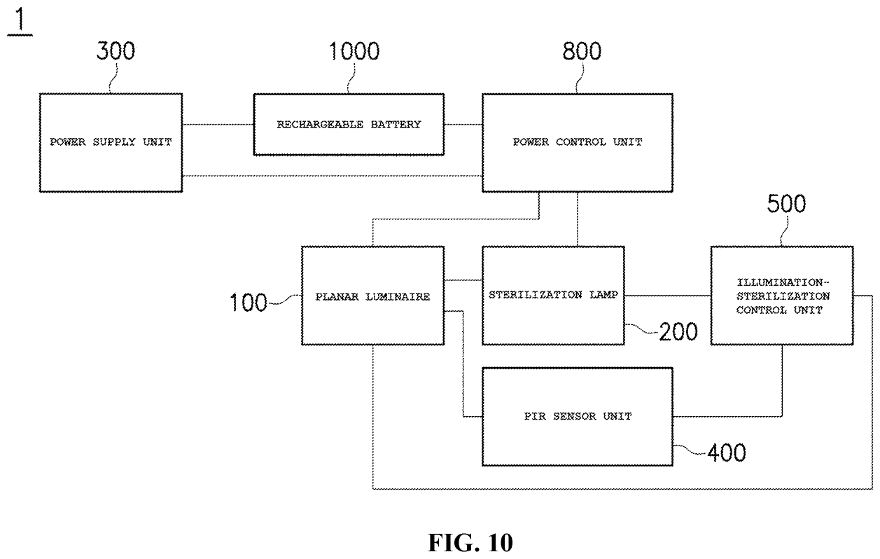

The sterilizer with planar lighting 1 according to the embodiment of the present invention may have a structure in which electric power is supplied to the sterilization lamp 200 by a rechargeable battery 1000 as illustrated in FIG. 10. For this purpose, the sterilizer with planar lighting 1 may include the rechargeable battery 1000 and the power control unit 800 to perform power control for the planar luminaire 100 and the sterilization lamp 200.

The rechargeable battery 1000 is connected to the power supply unit 300 for charging, and is connected to the sterilization lamp 200 for supply of electric power thereto. The sterilization lamp 200 may be directly connected to the power supply unit 300 to receive electric power.

The power control unit 800 is connected to the power supply unit 300 and the rechargeable battery 1000 to control power supply. In particular, the power control unit 800 according to the embodiment of the present invention, when the planar luminaire 100 is turned on, induces charging of the rechargeable battery 1000 while cutting off supply of electric power to the sterilization lamp 200 from the rechargeable battery 1000 and the power supply unit 300. This may prevent the sterilization lamp 200 from being turned on in a state in which an object is present in the illumination target region due to maloperation of the sterilization lamp switch 4 or malfunction/error of the device by the user.

The power control unit 800 according to the embodiment of the present invention, when the planar luminaire 100 is turned off, applies electric power to the sterilization lamp 200 from one of the rechargeable battery 1000 and the power supply unit 300. This enables the sterilization lamp 200 to be turned on after the planar luminaire 100 is turned off, regardless of the operation of the luminaire switch 3 and the sterilization lamp switch 4.

The PIR sensor unit 400 performs real-time infrared detection of the illumination target region 2, and generates object recognition information indicating that a person or an animal is present in the illumination target region 2. That is, when a person or an animal is present in the illumination target region 2, object recognition information is generated. On the other hand, when a person or an animal is not present in the illumination target region 2, object recognition information is not generated.

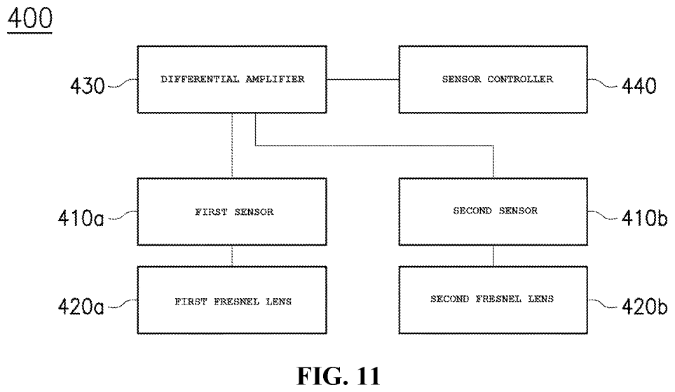

As illustrated in FIGS. 2 and 11, the PIR sensor unit 400 according to the embodiment of the present invention includes a first sensor 410a, a second sensor 410b, a first Fresnel lens 420a, a second Fresnel lens 420b, a differential amplifier 430, and a sensor controller 440. The first sensor 410a, the second sensor 410b, the first Fresnel lens 420a, the second Fresnel lens 420b, the differential amplifier 430, and the sensor controller 440 are fixedly arranged on a sensor fixing frame 450. The sensor fixing frame 450 may be formed integrally with the planar luminaire 100 or the sterilization lamp 200 and fixedly installed on the wall surface (ceiling surface, etc.) of the residential or office space, or may be separated from the planar luminaire 100 or the sterilization lamp 200 and fixedly installed on the wall surface (ceiling surface, etc.) of the residential or office space. The sensor fixing frame 450 may have an elongated rectangular plate shape, but the present invention is not limited thereto.

The first sensor 410a is fixed at a set position, and has a structure in which a pair of pyroelectric sensor elements 411 placed in an elongated straight shape in a reference direction are spaced parallel to each other and connected in series.

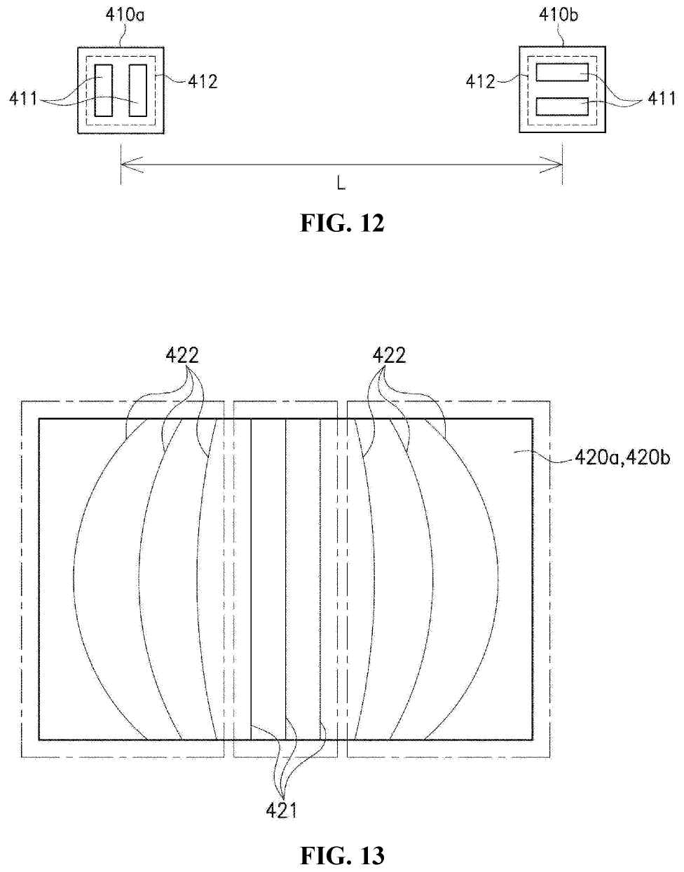

As illustrated in FIG. 12, the second sensor 410b is fixed at a position spaced apart from the first sensor 410a by a set distance L, and has a structure in which a pair of pyroelectric sensor elements 411 placed in an elongated straight shape in a direction perpendicular to the reference direction are spaced parallel to each other and connected in series.

The set distance L between the first sensor 410a and the second sensor 410b is set to be less than or equal to ½ of the infrared detection distance L of the PIR sensor unit 400.

The first Fresnel lens 420a is disposed in communication with a transmission window 412 of the first sensor 410a to guide infrared light generated in the illumination target region 2 to the transmission window 412. The second Fresnel lens 420b is disposed in communication with a transmission window 412 of the second sensor 410b to guide infrared light generated in the illumination target region 2 to the transmission window 412.

As illustrated in FIG. 13, the first Fresnel lens 420a and the second Fresnel lens 420b according to the embodiment of the present invention has a structure in which, in a direction in which the pyroelectric sensor elements 411 of each of the first sensor 410a and the second sensor 410b are placed in an elongated straight shape, one or more linear refractive grooves 421 are formed in the central portion thereof and curved refractive grooves 422 whose curvatures increase radially (r) outwardly are formed in the left and right portions thereof.

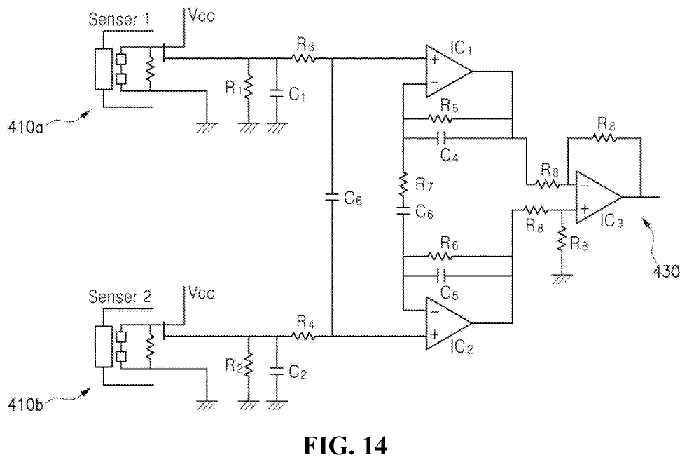

As illustrated in FIG. 14, the differential amplifier 430 is connected to the first sensor 410a and the second sensor 410b, and differentially measures and amplifies the output signal of the first sensor 410a and the output signal of the second sensor 410b to perform a band-pass filter function.

The sensor controller 440 receives signals from the differential amplifier 430 to generate object recognition information.

The PIR sensor unit 400 according to the embodiment of the present invention is implemented as a circuit configuration illustrated in FIG. 14. The differential amplifier 430 differentially measures signals in the state in which the pyroelectric sensor elements 411 in the first sensor 410a and the pyroelectric sensor elements 411 in the second sensor 410b are arranged at 90 degrees, the first and second sensors 410a and 410b being spaced apart from each other by the set distance L set to be less than or equal to ½ of the infrared detection distance L of the PIR sensor unit 400. The present invention employs: 1) removal of high frequency noise through R3, R4, and C3; 2) removal of high frequency noise through C1 and C2; 3) composition of instrumentation amplifiers by IC1, IC2, and IC3; 4) formation of low-pass filters by R5 and C4 and R6 and C5; 5) formation of a high-pass filters by R7 and C6; 6) induction of 16 times signal amplification by differential amplification at the rear end of the circuit by setting R5=R6 (gain=2*R5/R7, set to about 16 times); 7) a window comparator; and the like. Accordingly, the PIR sensor unit 400 according to the embodiment of the present invention may detect a fine movement of an object in a state in which noise is minimized, thereby ensuring improved object recognition accuracy and precision and thus high safety.

The illumination-sterilization control unit 500 is connected to the planar luminaire 100, the sterilization lamp 200, and the PIR sensor unit 400. The illumination-sterilization control unit 500 controls the operation of the sterilization lamp 200 by an automatic sterilization process when the object recognition information is not input from the PIR sensor unit 400, and deactivates the sterilization lamp 200 by a sterilization stop process when the object recognition information is input from the PIR sensor unit 400.

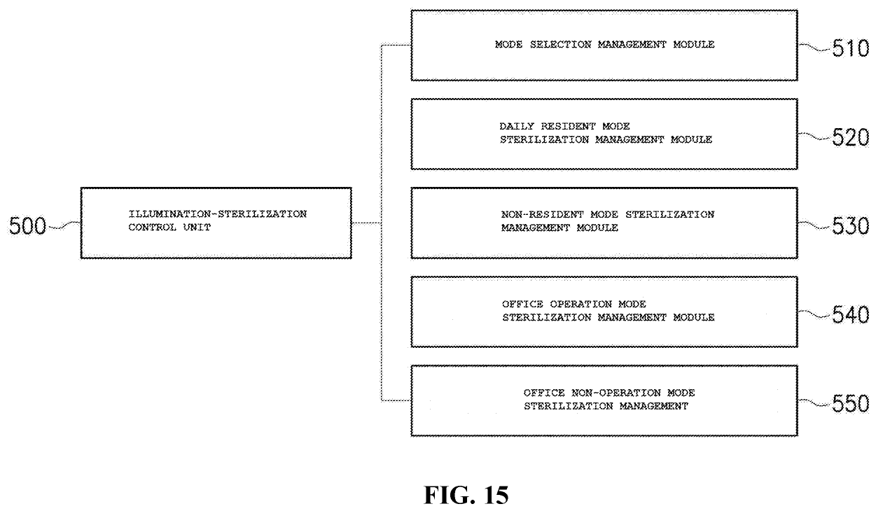

As illustrated in FIG. 15, the illumination-sterilization control unit 500 according to the embodiment of the present invention includes a mode selection management module 510, a daily resident mode sterilization management module 520, a non-resident mode sterilization management module 530, an office operation mode sterilization management module 540, and an office non-operation mode sterilization management module 550.

The mode selection management module 510 is a module configured to select and manage one of a daily resident mode, a non-resident mode, an office operation mode, and an office non-operation mode by an administrator.

The daily resident mode sterilization management module 520 is a module configured to turn on the sterilization lamp 200 after a set delay time from a start time at which object recognition information is not input in a time period during which the object recognition information is not input in the daily resident mode, and to turn off the sterilization lamp 200 after a set sterilization duration time has elapsed. When the object recognition information is input while the sterilization lamp 200 is in operation, the daily resident mode sterilization management module 520 immediately turns off the sterilization lamp 200.

The non-resident mode sterilization management module 530 is a module configured to turn on/off the sterilization lamp 200 at a set sterilization cycle in a time period in which object recognition information is not input in the non-resident mode. The sterilization cycle may be set by the administrator. When the object recognition information is input while the sterilization lamp 200 is in operation, the non-resident mode sterilization management module 530 immediately turns off the sterilization lamp 200.

The office operation mode sterilization management module 540 is a module configured to allow the sterilization lamp 200 to remain off in the office operation mode.

The office non-operation mode sterilization management module 550 is a module configured to turn on/off the sterilization lamp 200 at a set sterilization cycle in a time period in which object recognition information is not input in the office non-operation mode. The sterilization cycle may be set by the administrator. When the object recognition information is input while the sterilization lamp 200 is in operation, the office non-operation mode sterilization management module 550 immediately turns off the sterilization lamp 200.

The illumination-sterilization control unit 500 according to the embodiment of the present invention deactivates the daily resident mode sterilization management module 520, the non-resident mode sterilization management module 530, and the office non-operation mode sterilization management module 550 while the planar luminaire 100 is in operation. In addition, the illumination-sterilization control unit 500 according to the embodiment of the present invention, when the planar luminaire 100 is not in operation, deactivates the office operation mode sterilization management module 540 and activates the daily resident mode sterilization management module 520 in the office operation mode.

As described above, since the sterilization lamp is automatically turned on only when no object such as a person or a companion animal is present in the illumination target region in the sterilizer with planar lighting 1 according to the embodiment of the present invention, it is possible to perform sterilization in a safety-secured state to maintain the residential/office space in a sanitary state. In particular, the sterilizer with planar lighting 1 according to the embodiment of the present invention can perform power supply control and operation control according to various environmental conditions and situations by connecting the planar luminaire and the sterilization lamp to the SMPS through the switching operation unit and allowing the switching operation unit to selectively perform the simultaneous power supply to the planar luminaire and the sterilization lamp, the individual power supply thereto, the connection with the central control server, the individual connection with the luminaire switch/sterilization lamp switch, etc.

Although the sterilizer with planar lighting according to the embodiment of the present invention has been described above and illustrated in the drawings, it will be understood that this is provided by way of example only. It will be understood by those skilled in the art that various changes and modifications may be made without departing from the spirit and scope of the invention as defined in the following claims.

1. A sterilizer with planar lighting, comprising
a planar luminaire (100) configured to irradiate an illumination target region (2) with light by exposing a light projection surface (110) having a set area;
a sterilization lamp (200) configured to perform sterilization capability by irradiating the illumination target region (2) with ultraviolet light;
a power supply unit (300) configured to provide electric power to operate the planar luminaire (100) the sterilization lamp (200);
a PIR sensor unit (400) configured to perform real-time infrared detection of the illumination target region (2) and to generate object recognition information indicating that a person or an animal is present in the illumination target region (2);
an illumination-sterilization control unit (500) connected to the planar luminaire (100), the sterilization lamp (200), and the PIR sensor unit (400), and configured to control operation of the sterilization lamp (200) by an automatic sterilization process when the object recognition information is not input from the PIR sensor unit (400) and to deactivate the sterilization lamp (200) by a sterilization stop process when the object recognition information is input from the PIR sensor unit (400); and
a sterilization lamp-sensor assembly (1200) comprising:
a lower housing (1220) fixedly inserted into a mounting space for fitting (1110) of a frame (1100) formed integrally with the planar luminaire (100) around the planar luminaire (100), the mounting space (1110) being recessed to a set depth, the frame (1100) having two parallel sides;
an upper housing (1210) detachably coupled to the lower housing (1220) and having an open surface (1211) and a sensor through-hole (1212) formed on an upper surface thereof exposed to the outside;
an LED-sensor substrate (1250) fixed inside the lower housing (1220), in which case a plurality of sterilization LEDs (210) are arranged beneath the open surface (1211) of the upper housing (1210) and the PIR sensor unit (400) is disposed beneath the sensor through-hole (1212); and
a quartz glass plate (1260) disposed in communication with the open surface (1211) of the upper housing (1210) to transmit ultraviolet light from the sterilization LEDs (210), the sterilization lamp-sensor assembly (1200) being fitted in a detachable assembly structure into the mounting space (1110),
wherein the PIR sensor unit (400) consists of a PIR sensor unit (400), which is installed on one side of the frame and comprises a first sensor (410a) having a structure in which a pair of elongated straight pyroelectric sensor elements (411) are spaced parallel to each other and connected in series, and a PIR sensor unit (400) which is installed on the other side of the frame and comprises a second sensor (410b) having a pair of pyroelectric sensor elements (411) formed parallel to each other in a direction perpendicular to the installation direction of the pyroelectric sensor elements (411) of the first sensor (410a).

2. The sterilizer with planar lighting according to claim 1, wherein the illumination-sterilization control unit (500) comprises:
a mode selection management module (510) configured to select one from a group consisting of a daily resident mode, a non-resident mode, an office operation mode, and an office non-operation mode by an administrator;
a daily resident mode sterilization management module (520) configured to turn on the sterilization lamp (200) after a set delay time from a start time at which the object recognition information is not input in a time period during which the object recognition information is not input in the daily resident mode, and to turn off the sterilization lamp (200) after a set sterilization duration time has elapsed, the daily resident mode sterilization management module (520) being configured to immediately turn off the sterilization lamp (200) when the object recognition information is input while the sterilization lamp (200) is in operation;
a non-resident mode sterilization management module (530) configured to turn on/off the sterilization lamp (200) at a set sterilization cycle in a time period in which the object recognition information is not input in the non-resident mode, the non-resident mode sterilization management module (530) being configured to immediately turn off the sterilization lamp (200) when the object recognition information is input while the sterilization lamp (200) is in operation, the sterilization cycle being set by the administrator;
an office operation mode sterilization management module (540) configured to allow the sterilization lamp (200) to remain off in the office operation mode; and
an office non-operation mode sterilization management module (550) configured to turn on/off the sterilization lamp (200) at a set sterilization cycle in a time period in which the object recognition information is not input in the office non-operation mode, the office non-operation mode sterilization management module (550) being configured to immediately turn off the sterilization lamp (200) when the object recognition information is input while the sterilization lamp (200) is in operation, the sterilization cycle being set by the administrator.

3. The sterilizer with planar lighting according to claim 1, comprising:
an SMPS (600) connected to the power supply unit (300);
a switching operation unit (700) connected to the SMPS (600), and configured to implement a plurality of switching operation modes for applying electric power to the planar luminaire (100) and the sterilization lamp (200); and
a power control unit (800) configured to control implementation of the switching operation modes of the switching operation unit (700),
wherein the switching operation unit (700) comprises:
a simultaneous connection switching module (710) configured to induce anti-virus planar lighting by connecting the planar luminaire (100) and the sterilization lamp (200) to the SMPS (600) at the same time; and
an individual connection switching module (720) configured to connect each of the planar luminaire (100) and the sterilization lamp (200) to the SMPS (600) individually, to induce implementation of planar lighting capability or sterilization capability by user's arbitrary selection.

4. The sterilizer with planar lighting according to claim 1, further comprising:
a rechargeable battery (1000) connected to the power supply unit (300) for charging and connected to the sterilization lamp (200) for supply of electric power thereto; and
a power control unit (800) connected to the power supply unit (300) and the rechargeable battery (1000) to control power supply, the power control unit (800) being configured, when the planar luminaire (100) is turned on, to induce charging of the rechargeable battery (1000) while cutting off supply of electric power to the sterilization lamp (200) from the rechargeable battery (1000) and the power supply unit (300), the power control unit (800) being configured, when the planar luminaire (100) is turned off, to apply electric power to the sterilization lamp (200) from one of the rechargeable battery (1000) and the power supply unit (300),
wherein the sterilizer with planar lighting enables the sterilization lamp (200) to be turned on after the planar luminaire (100) is turned off, regardless of operation of a luminaire switch (3) and a sterilization lamp switch (4).
